(19) United States
(12) Patent Application Publication
(10) Pub. No.: US 2013/0123626 A1
(43) Pub. Date: May 16, 2013

(54) ULTRASOUND APPARATUS FOR DISPLAYING A PLURALITY OF KEY-SETS AND ULTRASOUND DIAGNOSIS METHOD USING THE ULTRASOUND APPARATUS

(71) Applicant: Samsung Medison Co., Ltd., Gangwon-do (KR)

(72) Inventors: Kyeong-gu WOO, Gangwon-do (KR); Hak-il KANG, Gangwon-do (KR); Jae-moon JO, Gangwon-do (KR)

(73) Assignee: Samsung Medison Co., Ltd., Gangwon-do (KR)

(21) Appl. No.: 13/676,907

(22) Filed: Nov. 14, 2012

(30) Foreign Application Priority Data
Nov. 16, 2011 (KR) 10-2011-0119783

(51) Int. Cl. A61B 8/00; A61B 8/13

(52) U.S. Cl. CPC A61B 8/467; A61B 8/463; A61B 8/13; A61B 8/466
USPC 600/440

(57) ABSTRACT

An ultrasound apparatus comprising a control panel on which a plurality of key-sets are printed, wherein each of the plurality of key-sets responds to at least one beam from among a plurality of beams, and then is displayed on the control panel.

### CROSS-REFERENCE TO RELATED PATENT APPLICATION

This application claims the benefit of Korean Patent Application No. 10-2011-0119783, filed on Nov. 16, 2011, in the Korean Intellectual Property Office, the disclosure of which is incorporated herein in its entirety by reference.

### BACKGROUND OF THE INVENTION

1. Field of the Invention

The present invention relates to an ultrasound apparatus for selectively displaying one of various key-sets on a control panel by controlling beams emitted from a light source, and an ultrasound diagnosis method using the ultrasound apparatus.

2. Description of the Related Art

An ultrasound diagnosis apparatus delivers an ultrasound signal from a body surface of a target object to a predetermined part in the body, and then obtains an image of tomography or hematocele of a soft tissue by using information of the ultrasound signal reflected from a tissue in the body.

The ultrasound diagnosis apparatus is advantageous in that it is small, is inexpensive, and displays in real-time. Also, the ultrasound diagnosis apparatus is not harmful such as radioactivity and thus is safe, so that it is widely used together with an X-ray diagnosis apparatus, a computerized tomography (CT) scanner, a magnetic resonance image (MRI) apparatus, a nuclear medicine diagnosis apparatus, and the like.

In general, the ultrasound diagnosis apparatus has a plurality of buttons for controlling various functions. The plurality of buttons are mounted in the ultrasound diagnosis apparatus via a plurality of button print processes, such that a defect rate of the ultrasound diagnosis apparatus is high.

Also, since the buttons of the ultrasound diagnosis apparatus are printed in several languages for export, there are many difficulties in stock management.

Therefore, it is required to provide a system capable of decreasing the number of button print processes for the ultrasound diagnosis apparatus, and efficiently performing stock management with respect to ultrasound diagnosis apparatuses that are printed in several languages.

### SUMMARY OF THE INVENTION

The present invention provides an ultrasound apparatus for displaying at least one of a plurality of key-sets on a control panel by controlling beams emitted from a light source, and an ultrasound diagnosis method using the ultrasound apparatus. According to an aspect of the present invention, there is provided an ultrasound apparatus including a light source for emitting a plurality of beams; a control panel on which a plurality of key-sets are printed; and a control unit for controlling the light source to emit at least one beam to the control panel, wherein each of the plurality of key-sets responds to at least one beam from among the plurality of beams emitted from the light source, and then is displayed on the manipulation unit.

The plurality of key-sets may be printed on the manipulation unit by a special ink that responds to at least one beam from among the plurality of beams.

The plurality of beams may be classified according to colors or frequency characteristics.

The plurality of key-sets may correspond to a plurality of languages, respectively, or may correspond to a plurality of image modes, respectively.

The plurality of image modes may include an ultrasound image mode, and the ultrasound image mode may include at least one of a brightness mode (B mode), a color mode (C mode), a motion mode (M mode), a pulsed-wave (PW) mode, a continuous wave (CW) mode, a two-dimensional (2D) mode, and a three-dimensional (3D) mode.

The control unit may control the light source to emit at least one beam from among the plurality of beams to the control panel, according to a language or an image mode selected by a user.

According to another aspect of the present invention, there is provided an ultrasound diagnosis method including operations of receiving selection information from an external source, wherein the selection information is used for selection of a predetermined language or a predetermined image mode; and selecting at least one language or at least one image mode according to the selection information and controlling a color or a frequency of a beam emitted from a light source so as to allow a key-set to be displayed on a control panel, wherein the key-set corresponds to the at least one language or the at least one image mode.

According to another aspect of the present invention, there is provided a computer-readable recording medium having recorded thereon a program for executing the ultrasound diagnosis method.

### BRIEF DESCRIPTION OF THE DRAWINGS

The above and other features and advantages of the present invention will become more apparent by describing in detail exemplary embodiments thereof with reference to the attached drawings in which:

FIG. 1 is a block diagram illustrating a structure of an ultrasound apparatus according to an embodiment of the present invention;

FIG. 2 illustrates a light source related to the embodiment of the present invention in FIG. 1;

FIG. 3 is a flowchart of an ultrasound diagnosis method, according to an embodiment of the present invention;

FIG. 4 is a diagram illustrating a plurality of key-sets that correspond to a plurality of languages, which are related to an embodiment of the present invention; and

FIG. 5 is a diagram illustrating a plurality of key-sets that correspond to a plurality of image modes, which are related to an embodiment of the present invention.

### DETAILED DESCRIPTION OF THE INVENTION

Terms or words used in the following description should not be construed as being limited to common or general meanings but should be construed as fully satisfying the concept of the invention. In the following description, the applicant arbitrarily selects some terms or words, and in those cases, the detailed meaning are provided here.

Also, when a part “includes” or “comprises” an element, unless there is a particular description contrary thereto, the part can further include other elements, not excluding the other elements. In the following description, terms such as “unit” and “module” indicate a unit for processing at least one function or operation, wherein the unit and the block may be embodied as hardware or software or embodied by combining hardware and software.

In the following description, “ultrasound image” indicates an image with respect to a target object, which is obtained by using ultrasound. The target object may indicate a part of a human body. For example, the target object may include human organs such as the liver, the heart, the womb, the brain, the breast, the abdomen, and the like, an unborn child, or the like.

In the following description, “user” may indicate a medical expert including a doctor, a nurse, a clinical pathologist, a medical image expert, or the like but is not limited thereto.

Hereinafter, the present invention will be described in detail by explaining exemplary embodiments of the invention with reference to the attached drawings. In the following description, functions or constructions that are not related to the present invention are omitted, and like reference numerals in the drawings denote like elements.

Expressions such as “at least one of,” when preceding a list of elements, modify the entire list of elements and do not modify the individual elements of the list.

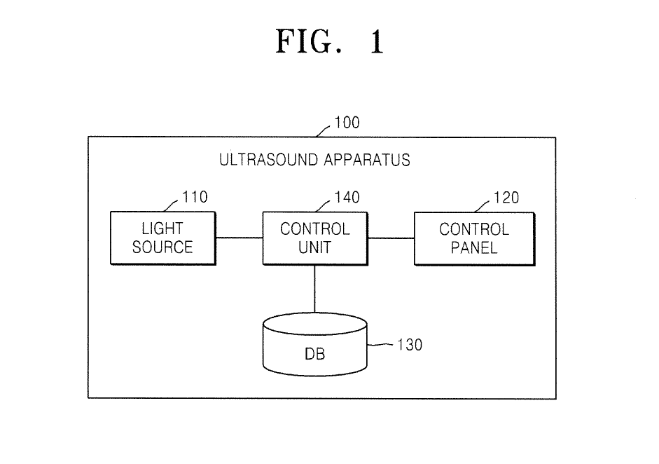

FIG. 1 is a block diagram illustrating a structure of an ultrasound apparatus 100 according to an embodiment of the present invention.

The ultrasound apparatus 100 indicates an apparatus that may obtain an ultrasound image with respect to a target object by using ultrasound and that may display the ultrasound image to a user.

The ultrasound apparatus 100 may be embodied in various forms. For example, the ultrasound apparatus 100 may be embodied as a fixed terminal or a mobile terminal. Examples of the mobile terminal may include a laptop computer, a person digital assistant (PDA), a tablet, or the like.

As illustrated in FIG. 1, the ultrasound apparatus 100 may include a light source 110, a control panel 120, a database (DB) 130, and a control unit 140. However, not every element of the ultrasound apparatus 100 is necessary. More elements or less elements than the aforementioned elements may embody the ultrasound apparatus 100.

The light source 110 may emit a plurality of beams. The plurality of beams may be classified according to colors or frequency characteristics. For example, the plurality of beams may include blue, red, green, yellow, purple, or the like.

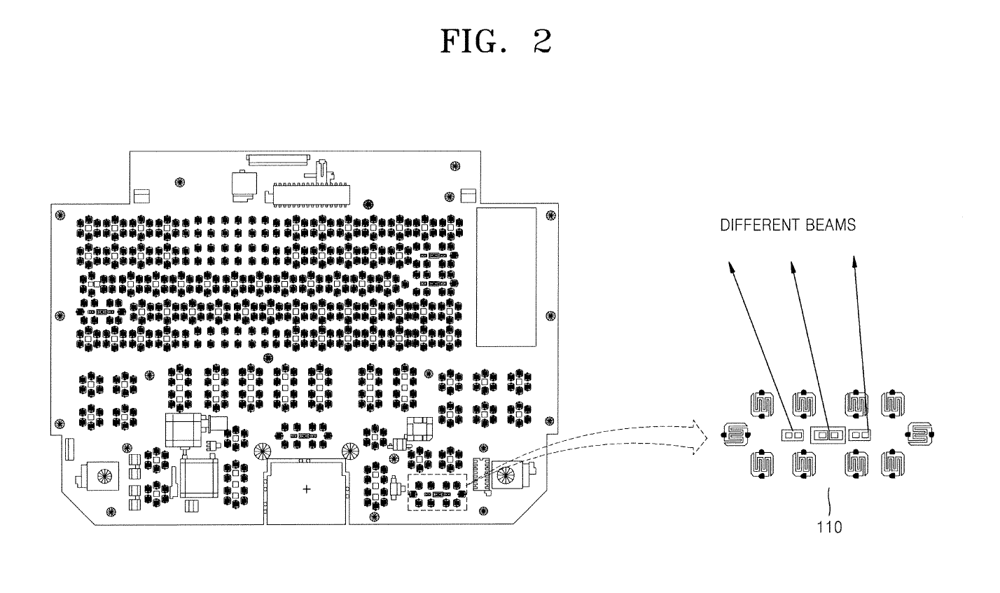

According to the present embodiment, as illustrated in FIG. 2, the light source 110 may be disposed in a predetermined region of a control panel (CP) board and may emit various types of beams. According to another embodiment, the light source 110 may separately exist outside of the ultrasound apparatus 100.

A plurality of key-sets may be printed on the control panel 120. In the present embodiment, the control panel 120 may be a button type control panel (i.e., a user input unit) and may be formed as a key pad.

The key-set indicates a group of input keys that are required to express a particular language or to control a particular image mode. The plurality of key-sets in the present embodiment may correspond to a plurality of languages, respectively, or a plurality of image modes, respectively.

For example, a key-set for the Korean language may include [symbol] [symbol] [symbol] [symbol] [symbol] [symbol] and |, a key-set for the English language may include a, b, c, d, e, f, g, h, i, j, k, l, m, n, o, p, q, r, s, t, u, v, w, x, y, and z, and a key-set for the Japanese language may include [symbol] [symbol] [symbol] [symbol] [symbol] [symbol] [symbol] [symbol] [symbol] [symbol] [symbol] [symbol] [symbol] and [symbol] .

According to the present embodiment, the key-set corresponding to the particular image mode may be a key-set for functions or menus that are the most-frequently-used in all image modes.

Each of the plurality of key-sets may respond to at least one beam from among the plurality of beams emitted from the light source 110 and thus may be displayed on the control panel 120.

For example, if a beam emitted from the light source 110 is a blue beam, a key-set for the Korean language may respond, and if a beam emitted from the light source 110 is a red beam, a key-set for the English language may respond.

According to the present embodiment, the plurality of key-sets may be printed on the control panel 120 by a special ink. The special ink may allow the plurality of key-sets to respond to at least one beam. That is, according to the present embodiment, special inks that respond to particular beams, respectively, are sprayed on the control panel 120 a plurality of times, so that the plurality of key-sets that are used for various languages or various modes may be selectively displayed on the control panel 120.

According to the present embodiment, in a case where a language or an image mode used in the ultrasound apparatus 100 is added, a key-set may be additionally printed on the control panel 120.

An image mode according to the present embodiment may include an ultrasound image mode. The ultrasound image mode may be at least one of a brightness mode (B mode), a color mode (C mode), a motion mode (M mode), a pulsed-wave (PW) mode, a continuous wave (CW) mode, a two-dimensional (2D) mode, and a three-dimensional (3D) mode. Alternatively, the ultrasound image mode may be a composite mode formed by combining at least two image modes.

The DB 130 may store information about a key-set. For example, the DB 130 may store information about the key-set corresponding to the particular language or the key-set corresponding to the particular image mode.

The DB 130 may also store information about languages or information about image modes, which may be used or set in the ultrasound apparatus 100. Also, the DB 130 may store information about keys that are frequently used by a user in the particular image mode.

The DB 130 may store a predetermined key-set and information about at least one beam corresponding to the predetermined key-set.

According to the present embodiment, the DB 130 may be included in the ultrasound apparatus 100 or may exist in the form of an external memory. Also, the DB 130 may be a storage server that is embodied on web.

The control unit 140 may control all operations of the light source 110, the control panel 120, and the DB 130. According to the present embodiment, the control unit 140 may control the light source 110 to emit at least one beam to the control panel 120.

The control unit 140 may receive selection information from an external source so as to select a predetermined language or a predetermined image mode. In this case, the control unit 140 may control the light source 110 to emit at least one beam from among the plurality of beams to the control panel 120 according to the selection information.

For example, when a user selects a language to be applied to the ultrasound apparatus 100, the control unit 140 may control a beam emitted from the light source 110 so as to allow a key-set, which corresponds to the selected language, to be displayed on the control panel 120.

According to another embodiment, when the user selects at least one image mode from among a plurality of ultrasound image modes, the control unit 140 may control a color or a frequency of a beam emitted from the light source 110 so as to allow a key-set, which corresponds to the selected image mode, to be displayed on the control panel 120.

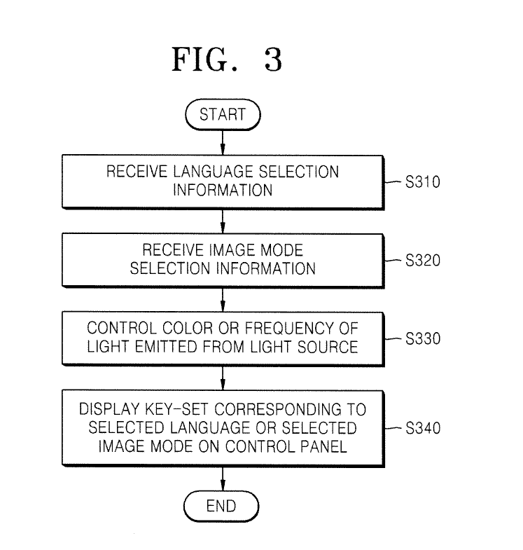

Hereinafter, an ultrasound diagnosis method of displaying a plurality of key-sets corresponding to a plurality of languages or a plurality of ultrasound image modes, by using each element of the ultrasound apparatus 100, is described in detail with reference to FIG. 3.

FIG. 3 is a flowchart of an ultrasound diagnosis method according to an embodiment of the present invention.

As illustrated in FIG. 3, the method may include an operation of receiving language selection information from an external source so as to select a predetermined language (operation S310). That is, a user may select a language to be used in the ultrasound apparatus 100.

The language selection information may include selection information with respect to the Korean language, the English language, the Chinese language, the

Japanese language, the French language, the German language, the Russian language, or the like but is not limited thereto.

The method may include an operation of receiving image mode selection information from an external source so as to select a predetermined image mode (operation S320). The user may select an image mode to be used in the ultrasound apparatus 100.

The image mode may include an ultrasound image mode, and the ultrasound image mode may include at least one of a B mode, a C mode, an M mode, a PW mode, a CW mode, a 2D mode, and a 3D mode.

Thus, according to the present embodiment, the image mode selection information may include selection information about at least one of the B mode, the C mode, the M mode, the PW mode, the CW mode, the 2D mode, and the 3D mode.

According to the present embodiment, the image mode selection information may include selection information about functions or menus that are frequently used in the ultrasound apparatus 100.

According to the present embodiment, operations S310 and S320 may be selectively performed. That is, the ultrasound apparatus 100 may receive only the language selection information or only the image mode selection information. Also, the ultrasound apparatus 100 may receive both of the language selection information and the image mode selection information.

For example, a user may select the Korean language as a language to be applied to the ultrasound apparatus 100. Also, the user may select the 3D mode so as to obtain a 3D ultrasound image. In this case, the user may select both the Korean language and a 3D image.

According to the present embodiment, the ultrasound apparatus 100 may control a color or a frequency of a beam emitted from the light source 110 so as to allow a key-set, which corresponds to the selection information, to be displayed on the control panel 120 (operation S330).

For example, it is assumed that a key-set corresponding to the Korean language responds to a blue color, a key-set corresponding to the English language responds to a red color, and a key-set corresponding to the Japanese language responds to a green color.

If the user selects the Korean language as a language to be applied to the ultrasound apparatus 100, the control unit 140 may control the light source 110 to emit a blue beam to the control panel 120 from among a plurality of beams. Also, if the user selects the English language, the control unit 140 may control the light source 110 to emit a red beam.

In another embodiment, it is assumed that a key-set corresponding to the B mode responds to a purple color, a key-set corresponding to the C mode responds to a yellow color, and a key-set corresponding to the M mode responds to an orange color.

If the user selects the B mode, the control unit 140 may control the light source 110 to emit a purple beam from among the plurality of beams to the control panel 120. Also, if the user selects the C mode, the control unit 140 may control the light source 110 to emit a yellow beam.

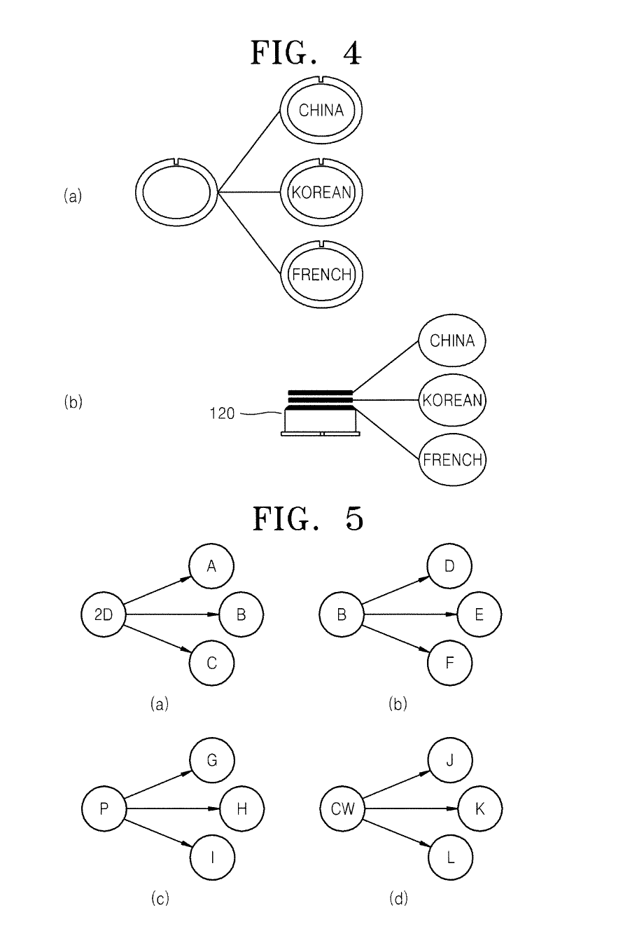

According to the present embodiment, a key-set corresponding to the selected language may be displayed on the control panel 120 (operation S340). For example, as illustrated in FIG. 4, it is assumed that the Chinese language, the Korean language, and the French language may be printed on the control panel 120, and a user selects the Chinese language. In this case, the light source 110 may emit a beam to which a key-set corresponding to the Chinese language responds, and then the key-set corresponding to the Chinese language may be displayed on the control panel 120.

If the user changes a language of the ultrasound apparatus 100 from the Chinese language to the Korean language, the control unit 140 controls the light source 110 to emit a beam to the control panel 120, wherein a key-set corresponding to the Korean language may respond to the beam. Here, the key-set corresponding to the Korean language may be displayed on the control panel 120.

Thus, the ultrasound apparatus 100 may efficiently display a plurality of languages by using the least number of buttons.

According to another embodiment, a key-set corresponding to a selected image mode may be displayed on the control panel 120 (operation S340). For example, as illustrated in FIG. 5, when the 2D mode is selected, a key-set including A, B, and C corresponding to the 2D mode may be displayed on the control panel 120, when the B mode is selected, a key-set including D, E, and F corresponding to the B mode may be displayed on the control panel 120, and when the CW mode is selected, a key-set including J, K, and L corresponding to the CW mode may be displayed on the control panel 120.

Therefore, according to the other embodiment, the ultrasound apparatus 100 may efficiently display a plurality of key-sets, which correspond to a large number of image modes, respectively, by using a small number of buttons.

According to the one or more embodiments of the present invention, the ultrasound diagnosis method can also be embodied as computer-readable codes on a computer-readable recording medium. The computer-readable recording medium is any data storage device that can store data which can be thereafter read by a computer system. Examples of the computer-readable recording medium include read-only memory (ROM), random-access memory (RAM), CD-ROMs, magnetic tapes, floppy disks, optical data storage devices, etc. Also, the computer may include the control unit 140 of the ultrasound apparatus 100.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. An ultrasound apparatus comprising a control panel on which a plurality of key-sets are printed,
wherein each of the plurality of key-sets responds to at least one beam from among a plurality of beams, and then is displayed on the control panel.

2. The ultrasound apparatus of claim 1, further comprising:
a light source for emitting the plurality of beams; and
a control unit for controlling the light source to emit at least one beam to the control panel.

3. The ultrasound apparatus of claim 1, wherein the plurality of key-sets are printed on the control panel by a special ink that responds to at least one beam from among the plurality of beams.

4. The ultrasound apparatus of claim 1, wherein the plurality of beams are classified according to colors or frequency characteristics.

5. The ultrasound apparatus of claim 1, wherein the plurality of key-sets correspond to a plurality of languages, respectively.

6. The ultrasound apparatus of claim 1, wherein the plurality of key-sets correspond to a plurality of image modes, respectively.

7. The ultrasound apparatus of claim 6, wherein the plurality of image modes comprise an ultrasound image mode.

8. The ultrasound apparatus of claim 7, wherein the ultrasound image mode comprises at least one of a brightness mode (B mode), a color mode (C mode), a motion mode (M mode), a pulsed-wave (PW) mode, a continuous wave (CW) mode, a two-dimensional (2D) mode, and a three-dimensional (3D) mode.

9. The ultrasound apparatus of claim 2, wherein the control unit controls the light source to emit at least one beam from among the plurality of beams to the control panel, according to a language or an image mode selected by a user.

10. An ultrasound diagnosis method comprising:
receiving selection information from an external source, wherein the selection information is used for selection of a predetermined language or a predetermined image mode; and
selecting at least one language or at least one image mode according to the selection information and controlling a color or a frequency of a beam emitted from a light source so as to allow a key-set to be displayed on a control panel, wherein the key-set corresponds to the at least one language or the at least one image mode.

11. The ultrasound diagnosis method of claim 10, wherein the at least one image mode comprises an ultrasound image mode.

12. The ultrasound diagnosis method of claim 11, wherein the ultrasound image mode comprises at least one of a brightness mode (B mode), a color mode (C mode), a motion mode (M mode), a pulsed-wave (PW) mode, a continuous wave (CW) mode, a two-dimensional (2D) mode, and a three-dimensional (3D) mode.

13. The ultrasound diagnosis method of claim 10, wherein the key-set indicates a plurality of key-sets that correspond to a plurality of languages or a plurality of image modes.

14. The ultrasound diagnosis method of claim 10, wherein the key-set is printed on the control panel by a special ink that responds to at least one beam from among a plurality of beams emitted from the light source.

15. A computer-readable recording medium having recorded thereon a program for executing the ultrasound diagnosis method of claim 10.
